(19) United States
(12) Patent Application Publication
(10) Pub. No.: US 2003/0007982 A1
(43) Pub. Date: Jan. 9, 2003

(54) Novel method for improving antioxidant status of animals consuming feeds contaminated with mycotoxins

(76) Inventors: Peter Surai, Scotland (GB); Julia Dvorska, Sumy (UA)

Correspondence Address: KING & SCHICKLI, PLLC, 247 NORTH BROADWAY, LEXINGTON, KY, 40507, US

(21) Appl. No.: 10/134,326

(22) Filed: Apr. 29, 2002

Related U.S. Application Data
(60) Provisional application No. 60/287,131, filed on Apr. 27, 2001.

(51) Int. Cl. A61K 35/72; A61K 33/06

(52) U.S. Cl. 424/195.16; 424/684

(57) ABSTRACT

Provided are compositions and methods to improve antioxidant status in vivo in animals comprising feeding an effective amount of a composition comprised of a modified yeast cell wall extract alone or in combination with a suitable mineral clay such as a zeolite, bentonite clay, or aluminum silicate to the animal to improve antioxidant status in vivo in animals, e.g., in animals consuming mycotoxin-contaminated feeds.

This application claims the benefit of priority in provisional application Serial No. 60/287,131 filed on Apr. 27, 2001.

### TECHNICAL FIELD

The present invention provides the surprising discovery that feeding a composition comprising a modified yeast cell wall extract alone or in combination with a clay such as a zeolite, bentonite, or other aluminosilicate clay has an in vivo antioxidant sparing effect in animals consuming it. In particular, the compositions of the invention are comprised of a modified yeast cell wall extract alone or in combination with aluminosilicate clay. In one embodiment, the modified yeast cell wall extract/clay composition is available from Alltech, Inc., Nicholasville, Ky.

The compositions described may be fed to any animal, including but not limited to human, avian, porcine, ruminant and equine species. When admixed with feed or fed as a supplement, the compositions result in surprising and unexpected improvements in antioxidant status in animals consuming them along with mycotoxin-contaminated foods or feeds, concomitantly improving performance and health and reducing incidence of diseases related to oxidative damage. The method comprises feeding an effective amount of a composition comprised of a yeast cell wall containing composition, e.g., a modified yeast cell wall extract, alone or in combination with a suitable clay, e.g., an aluminosilicate clay, as described above to an animal to improve the antioxidant status of the animal, e.g., by reducing the pro-oxidative effects of dietary mycotoxins.

### BACKGROUND OF THE INVENTION

Every year a substantial percentage of the world's grain and hay supply for animal feeds is contaminated by toxins produced by invading molds. Decreased feed nutritive value and instances of animal poisoning are most often traced to growth of various species of Aspergillus, Fuserium, and Penicillium in stored grain or other feeds. Mycotoxins affect feed nutritive value, livestock performance, and animal health. Mycotoxin-contaminated feeds are considerably less palatable to the animal, and the resulting decreased intake levels may exacerbate poor performance and/or toxicity problems.

The physiological effects of mycotoxins range from reduced feed intake and poor feed conversion to a general inability of an animal to thrive. Symptoms vary according to toxin. For example, zearalenone affects the reproductive organs of pigs and dairy cattle. Fumonisin causes a nervous disorder in horses due to its impact in the brain. Ochratoxin causes kidney damage. Aflatoxins, the most common mycotoxin, cause increased susceptibility to disease. At the organ or cellular level mycotoxins differ in their effects with severe damage done to the liver and kidney by aflatoxins and on reproductive organs by zearalenone.

The specific mechanism of action of mycotoxins on organs and cells has not been completely elucidated, but may be related to oxidative damage to tissues. As an example, the trichothecin toxin T-2, produced by Fusarium species, is known to be fat soluble. Accordingly, T-2 toxin can be incorporated into cell membranes and can change their structural and functional properties. T-2 toxin is also known to stimulate lipid peroxidation in the liver, resulting in increased production/release of free radicals with resulting tissue damage.

Free radicals are normally produced by cells in a number of normal metabolic processes such as prostaglandin and prostacyclin production, and during phagocytosis by neutrophils. The body has a number of defense mechanisms to limit the effect of the radicals to the sites where they are produced. These defense mechanisms include the activity of antioxidants such as vitamins A, C, and E, and the selenium-containing enzyme glutathione peroxidase.

There are circumstances, however, where the rate of free radical production exceeds the capacity of the defense systems to neutralize them, or where one or more of the defense systems is deficient. Additionally, conditions resulting in cell damage may result in release of free radicals in excess of the ability of the body's defense systems to neutralize them. Lipid peroxidation and damage to protein and DNA resulting from excessive free radical production have been implicated in the etiology of a wide variety of conditions including decreased immunocompetence, heart disease, cancer, rheumatoid arthritis and aging in humans, and a similarly wide-ranging group of pathological conditions in animals.

The formation of free radicals during lipid peroxidation can impair natural defense mechanisms, destroying critical nutrients such as vitamins A, D, and E, and resulting in impairment of vital metabolic functions. Rapid destruction of cellular components containing nucleic acids may occur, and organelle membranes may undergo peroxidative degeneration. The quality of products derived from affected animals may be also affected by the evolution of aldehydes and ketones which impart off-flavors to meat, and may undesirably alter skin pigmentation and egg yolk color (in poultry).

Studies have demonstrated the in vitro and in vivo binding effect of mannanoligosaccharides from yeast cell wall preparations and similar effects with mineral clays (See, e.g., Devegowda, G et al, “Mycotoxin Picture Worldwide: Novel Solutions For Their Counteraction”, Passport to the Year 2000, pp. 241-255, Nottingham Press 1998 (ISBN: 1-897676-662). Likewise, a limited amount of research has demonstrated a possible antioxidant effect of yeast cell wall preparations containing antioxidant vitamins and antioxidant enzymes in humans. See, e.g., Konig, D. et al, “Effect of 6-week nutritional intervention with enzymatic yeast cells and antioxidants on exercise stress and antioxidant status”, Wien Med Wochenschr 1999; 149(1): 13-8. See also, Tslapali, E. et al, “Glucans exhibit weak antioxidant activity, but stimulate macrophage free radical activity”, Free Radic Biol Med 2001 Feb; 30 (4): 393-402 and Krizkova, L. et al, “Antioxidative and antimutagenic activity of yeast cell wall mannans in vitro”, Muta Res 2001 Oct 18; 497 (1-2): 213-22. However, prior to the present invention, there has not been shown a direct in vivo systemic antioxidant effect of a yeast cell wall extract alone or in combination with a mineral clay

There is thus a need in the art for a composition and a method providing an antioxidant composition which exerts a direct in vivo effect as well as reducing the undesirable effects from excessive free radical production in vivo caused by mycotoxin consumption and the oxidative damage/lipid peroxidation resulting therefrom.

### SUMMARY OF THE INVENTION

In accordance with the purposes of the present invention as described herein, a novel method for reducing pro-oxidant effects of mycotoxins in animals is provided. In one aspect, the method of this invention comprises feeding a modified yeast cell wall extract alone or in combination with a mineral clay such as a zeolite or bentonite clay, or aluminum silicate to an animal to improve antioxidant status in vivo in animals consuming mycotoxin-contaminated foods or feeds.

In another aspect of this invention, a composition comprising a modified yeast cell wall extract alone or in combination with a mineral clay is provided which provides the surprising and unexpected effect of improving antioxidant status in animals consuming mycotoxin-contaminated food or feed.

Additional objects, advantages and other novel features of the invention will be set forth in part in the description that follows and in part will become apparent to those skilled in the art upon examination of the following or may be learned with the practice of the invention. To achieve the foregoing and other objects, and in accordance with the purposes of the present invention as described herein, a novel method is described for improving in vivo antioxidant status in animals, e.g., in animals consuming mycotoxin-contaminated foods or feeds.

In particular, the invention provides a method and a composition for improving antioxidant status in animals consuming mycotoxin-contaminated foods or feeds comprising a modified yeast cell wall extract. In another embodiment, the invention provides a method and a composition for improving antioxidant status in animals consuming mycotoxin-contaminated foods or feeds comprising a modified yeast cell wall extract and a suitable mineral clay, e.g., aluminosilicate. The yeast cell wall is extracted from a yeast organism which can be selected from among any of a number of yeasts, e.g., Saccharomyces cerevisiae. The aluminosilicate of the preferred embodiment is a standard commercial grade available from a variety of sources. In one embodiment, the yeast cell wall extract/aluminosilicate composition is available from Alltech, Inc., Nicholasville, Ky.

The compositions provided by this invention can be fed to any animal including, but not limited to, human, bovine, porcine, avian, equine, ovine, and caprine species. When admixed with feed or fed as a supplement, the compositions reduce the undesirable pro-oxidative effects of mycotoxins in animals consuming them, thereby improving performance and health.

### BRIEF DESCRIPTION OF THE DRAWINGS

FIG. 1 is a bar graph showing the extent of reduction of lipid peroxidation in the liver of animals consuming a diet comprised of the compositions of the invention.

### DETAILED DESCRIPTION OF THE INVENTION

The present invention is based upon the surprising discovery that a composition comprised of a yeast cell wall-derived extract alone or in combination with a mineral clay provides an unexpected improvement in antioxidant status in animals, e.g., in animals consuming mycotoxin-contaminated foods or feeds. Thus, the invention provides a method and a composition for improving antioxidant status utilizing such a yeast cell wall extract composition.

The yeast cell wall containing composition of the invention can be used in a method for improving the antioxidant status of an animal by exerting a direct in vivo antioxidant effect in the animal consuming the composition. The antioxidant effect is exacerbated in animals consuming mycotoxin contaminated feeds via the addition of a suitable mineral clay, e.g., aluminosilicate, to the composition comprised of the yeast cell wall containing composition. In one such embodiment, the yeast cell wall extract/aluminosilicate composition of the invention is described in U.S. Pat. No. 6,045,834, incorporated herein by reference.

An especially preferred embodiment of the compositions of the invention comprises from between 5% to about 7% aluminum silicate and between about 93% and about 95% yeast cell wall extract. The preferred physical form of the composition is a dry, free-flowing powder suitable for direct inclusion into animal feeds or as a supplement to a total mixed ration.

The compositions provided by the present invention can be utilized in methods of improving the in vivo antioxidant status of an animal by, e.g., directly incorporating an effective amount of the composition into commercially available feeds or fed as a supplement to commercially available feeds. When incorporated directly into animal feeds, the composition may be added to such feeds in amounts ranging from about 0.25 to about 4 kilograms per ton of feed. In a preferred embodiment, the compositions set forth herein are added to feeds in amounts ranging from 0.5 to about 3 kilograms per ton of feed. In an especially preferred embodiment, the composition is added to an animal feed in amounts ranging from 1 to 2 kilograms per ton of feed.

The composition contained in the present invention may be added to animal feedstuffs in amounts from about 0.0125% to 0.4% by weight of feed. In a preferred embodiment, the composition is added to animal feedstuffs in amounts from about 0.025% to 0.2% by weight of feed. In an especially preferred embodiment, the invention is added to animal feedstuffs in amounts from about 0.04% to 0.1% by weight of feed.

Alternatively, the compositions provided by the present invention may be directly fed to animals as a supplement in amounts ranging from 2.5 to 20 grams per animal per day. An especially preferred embodiment comprises feeding the composition provided by the present invention to animals in amounts ranging from 10 to 15 grams per animal per day. One of skill in the art can appreciate that the amount of the composition fed can vary depending upon the animal species, size of the animal and the type of feedstuff to which the composition is to be added.

The compositions of the present invention may be fed to any animal, including but not limited to, human, bovine, porcine, avian, equine, ovine, and caprine species. The methods of the invention comprise feeding the compositions of the invention to animals to improve antioxidant status, thereby increasing the overall health and performance of the animals. The in vivo antioxidants that are spared or otherwise affected by the methods of this invention include, but are not limited to the group consisting of Vitamin A, Vitamin E, Vitamin C, alpha-tocopherol, gamma-tocopherol, carotenoids, retinol, retinyl stearate, retinyl palmitate, retinyl oleate, retinyl linoleate, glutathione peroxidase and combinations thereof. The compositions of this invention further reduce susceptibility of tissues to lipid peroxidation in animals consuming them.

### EXAMPLES

The following examples are intended to be illustrative of the invention, and are not to be considered restrictive of the scope of the invention as otherwise described herein. The data set forth herein demonstrates the antioxidant-sparing ability of a composition comprised of a combination of modified yeast cell wall extract and aluminum silicate (MTB-100 or Mycosorb; Alltech, Inc., Nicholasville, Ky).

Quail (n=80) were separated into four experimental groups (n=20 each) according to dietary treatment: (1) control (basal diet); (2) T-2 toxin (as Fusarium sporotrichoides culture; 8.1 mg/kg of feed); (2) T-2 toxin plus hydrated aluminosilicate (ceolite; 30 g/kg of feed); and (4) T-2 toxin plus the modified yeast cell wall extract/aluminosilicate composition of this invention (1 g/kg of feed). After 30 days of feeding, the quail were sacrificed, and liver concentrations of antioxidants were measured. Liver samples were analyzed for concentrations of alpha- and gamma-tocopherols, total carotenoids, retinyl esters, free retinol, and ascorbic acid. Additionally, liver tissue susceptibility to lipid peroxidation was determined by measuring thiobarbituric acid reactive substances (TBARS) accumulation resulting from Fe-stimulated lipid peroxidation.

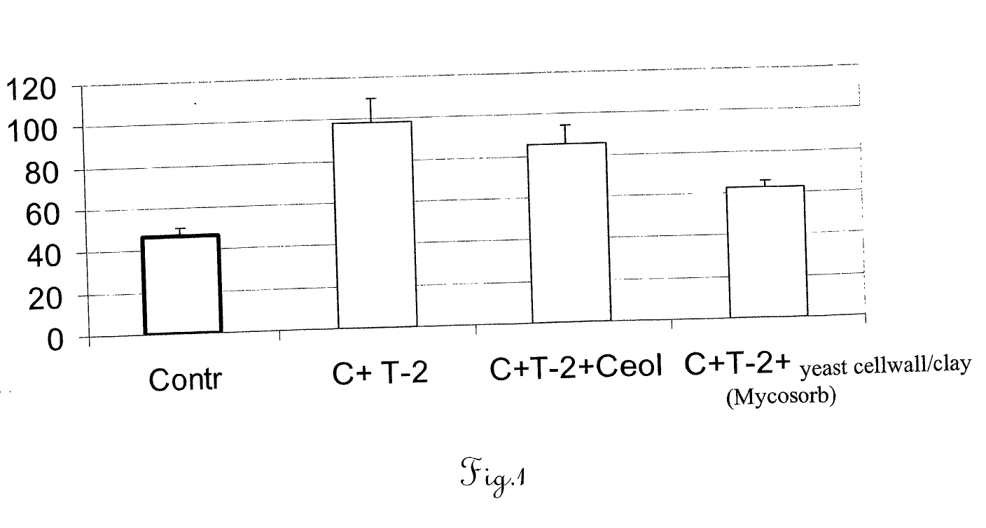

As best seen in Tables 1 and 2 below, T-2 toxin resulted in significant decreases in concentration of all antioxidants measured. For example, liver alpha- and gamma-tocopherol concentrations decreased by approximately 50% ( Table 1). Carotenoid concentrations in liver were reduced by 37%, and ascorbic acid concentration decreased by 39%. Concomitantly, as shown in FIG. 1, with the depletion of liver antioxidants, susceptibility of liver tissue to lipid peroxidation more than doubled.

TABLE 1
Antioxidants in the quail liver, μg/g (n = 5)

| | | | | T-2 + yeast | |
|---|---|---|---|---|---|
| | | | | cellwall/clay | |
| | Control | T-2 toxin | T-2 + ceolite | (Mycosorb) | P between |
| Antioxidant | 1 | 2 | 3 | 4 | 2 and 4 |
| α-tocopherol | 18.4 ± 1.33 | 10.2 ± 0.96 | 11.2 ± 1.03 | 14.6 ± 0.99 | P < 0.05 |
| γ-tocopherol | 1.9 ± 0.17 | 1.0 ± 0.09 | 1.2 ± 0.15 | 1.5 ± 0.07 | P < 0.01 |
| Carotenoids | 4.3 ± 0.13 | 2.7 ± 0.18 | 3.1 ± 0.30 | 3.6 ± 0.16 | P < 0.01 |
| Ascorbic Acid | 166.4 ± 8.6 | 101.1 ± 5.1 | 111.1 ± 4.8 | 150.6 ± 8.0 | P < 0.01 |

Addition of the yeast wall extract/aluminosilicate composition of this invention to the diets of quail fed T-2 toxin significantly reduced the alone. For example, the composition of this invention almost completely ameliorated the harmful effects of T-2 toxin on liver concentrations of Vitamin A metabolites (Table 2). Similarly, the composition of this invention significantly reduced the harmful effects of T-2 toxin on liver alpha- and gamma-tocopherol, carotenoids, and ascorbic acid (Table 1). Addition of the composition of this invention to the diets of quail also significantly improved resistance of liver tissue to lipid peroxidation (FIG. 1).

TABLE 2
Vitamin A in the quail liver, μg/g (n = 5)

| | | | | T-2 + yeast | |
|---|---|---|---|---|---|
| | | | | cellwall/clay | |
| | Control | T-2 toxin | T-2 + ceolite | (Mycosorb) | P between |
| Antioxidant | 1 | 2 | 3 | 4 | 2 and 4 |
| Retinol | 3.02 ± 0.21 | 2.07 ± 0.27 | 2.34 ± 0.26 | 3.01 ± 0.27 | P < 0.05 |
| Retinol-Stearate | 13.45 ± 1.09 | 6.92 ± 0.69 | 6.27 ± 0.54 | 9.61 ± 0.29 | P < 0.01 |
| Retinol-Palmitate | 27.81 ± 2.36 | 15.48 ± 1.17 | 18.59 ± 1.74 | 21.80 ± 2.22 | P < 0.01 |
| Retinol-oelate | 4.81 ± 0.42 | 3.04 ± 0.28 | 3.13 ± 0.43 | 5.04 ± 0.31 | P < 0.01 |
| Retinol-linolate | 3.72 ± 0.43 | 1.26 ± 0.21 | 2.15 ± 0.20 | 3.01 ± 0.34 | P < 0.01 |
| Total A | 52.81 ± 3.11 | 28.77 ± 1.49 | 32.47 ± 2.38 | 42.07 ± 2.12 | P < 0.01 |

In stark contrast ceolite, a hydrated aluminosilicate often added to animal diets for its alleged growth-promoting and mycotoxin-binding abilities, was significantly less efficacious in preventing antioxidant depletion and resistance to lipid peroxidation in livers of quail fed T-2 toxin. With the exception of retinol palmitate and retinyl linoleate (Table 2), there was virtually no difference in liver antioxidant status between quail fed T-2 toxin and those fed T-2 toxin plus ceolite. Similarly, addition of ceolite to the diets of quail fed T-2 toxin did not prevent the increases noted in susceptibility to lipid peroxidation (FIG. 1).

These results show that the compositions provided by the present invention, e.g., a modified yeast cell wall extract in combination with a suitable mineral clay, provides an effective method for improving in vivo antioxidant status in animals consuming mycotoxin-contaminated foods or feeds. When fed to animals exposed to mycotoxins in the diet, the present invention was effective at preventing depletion of natural antioxidants from the liver, and further resulted in reduced susceptibility to lipid peroxidation.

The foregoing description of a preferred embodiment of the invention has been presented for purposes of illustration and description. It is not intended to be exhaustive or to limit the invention to the precise form disclosed. Obvious modifications or variations are possible in light of the above teachings. The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitable entitled.

What is claimed is:

1. A method for improving the antioxidant status in an animal, comprising feeding to the animal an effective amount of a composition comprised of at least a portion of a yeast cell wall.

2. The composition of claim 1, wherein the portion of the yeast cell wall is comprised of a modified yeast cell wall extract.

3. The composition of claim 2, wherein the modified yeast cell wall extract is comprised of a modified yeast cell wall mannanoligosaccharide.

4. The composition of claim 1, wherein the animal is consuming or has consumed a feed containing mycotoxins and the composition further comprises a suitable mineral clay.

5. The composition of claim 4, wherein the suitable mineral clay is selected from the group consisting of a zeolite, a bentonite or an aluminosilicate or a combination thereof.

6. The method of claim 1, wherein the effective amount of the composition comprises from between about 0.0125% to between about 4% by weight of the animal's daily feed ration.

7. The method of claim 1, wherein the animal is selected from the group consisting of human, bovine, equine, ovine, porcine, avian and caprine species.

8. The method of claim 1, wherein the antioxidant status is improved by preventing the in vivo depletion of an antioxidant selected from the group consisting of Vitamin A, Vitamin E, Vitamin C, alpha-tocopherol, gamma-tocopherol, carotenoids, retinol, retinyl stearate, retinyl palmitate, retinyl oleate, retinyl linoleate, glutathione peroxidase and combinations thereof.

9. The method of claim 1, wherein the composition reduces tissue susceptibility to lipid peroxidation.

10. The method of claim 1, wherein the composition is admixed with any common animal feed prior to feeding.

11. The method of claim 1, whereby the composition is fed to any animal as a supplement to common feeds or forages.
